(19) United States
(12) Patent Application Publication
(10) Pub. No.: US 2026/0363906 A1
(43) Pub. Date: Nov. 19, 2026

(54) DEVICES FOR ADJUSTING THE INTRAOCULAR PRESSURE AND METHODS OF THEIR USE IN THE TREATMENT OF GLAUCOMA

(71) Applicant: GlauCure Ltd, Ra'anana (IL)

(72) Inventors: Gilad LITVIN, Ra'anana (IL); Almog ALEY-RAZ, Sde Warburg (IL)

(73) Assignee: GlauCure Ltd, Ra'anana (IL)

(21) Appl. No.: 19/470,889

(22) PCT Filed: Apr. 3, 2024
(86) PCT No.: PCT/IL2024/050343

§ 371 Date: Sep. 30, 2025

Related U.S. Application Data
(60) Provisional application No. 63/456,540, filed on Apr. 3, 2023.

(51) Int. Cl. A61F 9/00; A61F 9/007

(52) U.S. Cl. CPC A61F 9/0017; A61F 9/00781; A61F 2250/0013; A61F 2250/0024

(57) ABSTRACT

The invention provides implantable devices for controlling liquid outflow from an anterior chamber of an eye, kits comprising them and methods of their use in the treatment of glaucoma.

### BACKGROUND OF THE INVENTION

Glaucoma is an optic neuropathy characterized by acquired atrophy of the optic nerve and loss of retinal ganglion cells and their axons. Among other factors, elevated intraocular pressure contributes to progressive irreversible optic nerve damage and visual field loss, which may lead to complete blindness.

Worldwide, glaucoma is the second leading cause of blindness. Glaucoma affects 1 in 200 people aged fifty and younger, and 1 in 10 over the age of eighty. If the condition is detected early enough it is possible to stop the development or at least slow the progression of glaucoma with medical and surgical means.

Glaucoma is associated with increased pressure (intraocular pressure) of the liquid (aqueous humor) in the anterior chamber of the eye. There are many different sub-types of glaucoma but they can all be considered a type of optic neuropathy. Raised intraocular pressure is a significant risk factor for developing glaucoma (above 21 mmHg or 2.8 kPa). It is noted that nerve damage may develop due to increase in the intraocular pressure, however the magnitude of increased pressure that may cause nerve damage is individual, i.e. for certain people a relatively small increase in the intraocular pressure may result in irreversible nerve damage, while other people may have high eye pressure for long periods of time (i.e. months or years) before developing nerve damage. Untreated glaucoma leads to permanent damage of the optic nerve fibers and progressive visual field loss, which can lead to complete blindness.

Glaucoma can be divided roughly into two main categories, “open angle” glaucoma (OAG) and “closed angle” glaucoma (CAG). CAG can appear suddenly, leading to excruciating pain, or insidiously with minimal discomfort. In the acute form visual loss can progress quickly but the discomfort often leads patients to seek medical attention before permanent damage occurs. OAG and chronic angle closure glaucoma tends to progress at a slower rate and the patient may not notice that they have lost vision until the disease has progressed significantly.

The intraocular pressure is maintained by the dynamic equilibrium of aqueous production and outflow. The iris divides the anterior portion of the eye into anterior and posterior chambers, which communicate through the pupil. Aqueous humor, produced by the ciliary body, fills the posterior chamber, flows through the pupil into the anterior chamber, and leaves the eye through the trabecular meshwork, a connective tissue filter at the angle between the iris and the cornea. The aqueous humour passes through the trabecular meshwork into Schlemm's canal and into the episcleral venous system. Increased intraocular pressure is caused by obstruction to outflow. In OAG conditions, obstruction exists at a microscopic level in the trabecular meshwork. In CAG the iris obstructs the trabecular meshwork physically either because of anatomic variation leading to pupillary block and obstruction of aqueous humor flow into the anterior chamber, or by formation of adhesions between the iris and trabeculum.

There are a number of known devices intended to control the intraocular pressure in glaucomatotic eyes:

U.S. Pat. No. 5,300,020 discloses a surgically implantable device for controlled drainage flow of aqueous fluid from the anterior chamber of the eye into nearby sub-conjunctival space, for the relief of a glaucomatous condition of excessive pressure within the eye. The porous material in the device of U.S. Pat. No. 5,300,020 is indicated to be biodegradable, thus within a matter of time this material is decomposed, leaving a hollow tube connecting the anterior chamber with the near sub-conjunctival space. The purpose and effect of the biodegradability of the porous material inside the device is to avoid early hypotony. However, this device will not be able to improve surgical outcome since draining fluid to the near subconjuctival space will be able to relive intraocular pressure for a short period of time. With time scar tissue will develop in a percentage of patients thus clogging the device and conjunctival bleb.

U.S. Pat. No. 5,743,868 discloses a unitary, pressure-regulating corneal implant device for use in controlling intraocular pressure. This implant, having a conduit with a bore and a porous core material disposed in the bore, allows egress of aqueous humor from the anterior chamber of the eye. The conduit is elongated for extending from the ocular surface of the eye substantially flush therewith through the corneal stroma, and into the anterior chamber. This is an open system device, thus allowing egress of fluid into the eye possibly containing infections agents. Cornea will most probably reject the device as a foreign body (usually the case when implanting a body into the cornea) and also distorts the optical surface of the cornea thereby giving rise to optical aberrations to patients treated with this device.

U.S. Pat. No. 4,946,436 relates to a porous device for implantation in the scleral tissue of the eye to relieve the intraocular pressure of glaucoma and a method for surgically implanting the device. It is noted that such devices are intended for being implanted intrasclerally and thus will be able to remove the fluid only to the subsilliary space.

Devices described in U.S. Pat. No. 4,946,436 were not found to improve surgical results when compared with trabeculectomy, where treating glaucoma.

### SUMMARY OF THE INVENTION

The invention provides an implantable device for controlling liquid outflow from an anterior chamber of an eye, comprising: a tubular component comprising at least one tube; wherein said tubular component has an elongated body having a first end that is positionable (capable of being positioned) at an anterior chamber of the eye and a second end that is positionable (capable of being positioned) at an extraocular space and having an entry point through the sclera of said eye; wherein said tubular component is configured to provide a fluid path for draining fluid entering the first end of said tubular component from the anterior chamber to the extraocular space via the second end; wherein said tubular component is formed from at least non-porous non-degradable polymer; at least one patch component positionable (capable of being positioned) under the conjunctiva of the eye formed from at least one porous non-degradable polymer.

The invention provides an implantable device for the treatment of glaucoma comprising: a tubular component comprising at least one tube; wherein said tubular component has an elongated body having a first end that is positionable at an anterior chamber of the eye and a second end that is positionable at an extraocular space and having an entry point through the sclera of said eye; wherein said tubular component is configured to provide a fluid path for draining fluid entering the first end of said tubular component from the anterior chamber to the extraocular space via the second end; wherein said tubular component is formed from at least non-porous non-degradable polymer; at least one patch component positionable under the conjunctiva of the eye formed from at least one porous non-degradable polymer.

The device of the present invention allows for evacuating aqueous humor from the eye to the extraocular space (such as the retro bulbar/orbital/intraconal space). The extraocular space has positive pressure of about 3-6 mmHg. Previous solutions mentioned above drain the eye to the subconjunctival space that has negative pressure due to the hydrodynamics of the episcleral venous plexus that drains the anterior eye (Enz, T.J.; Tschopp, M. Assessment of Orbital Compartment Pressure: A Comprehensive Review. Diagnostics 2022, 12, 1481. https://doi.org/10.3390/diagnostics12061481).

Said tubular component has resistance and thus creates a lower threshold under which the extraocular (intraocular) pressure theoretically cannot drop to. This is achieved by manipulating the length and lumen diameter of said tubular component according to the Hagen-Poiseuille equation (giving the pressure drop in an incompressible and Newtonian fluid in laminar flow flowing through a long cylindrical pipe of constant cross section).

Said patch component allows for integration with the surrounding tissue under the conjunctiva. Said patch component is able to conceal the tubular component from the overlying conjunctiva thus hastening integration. Since said patch is non-degradable this allows forming it in a thickness that is thinner than tissue thickness. Furthermore, said patch component fixates the entire tubular component a specific and predetermined location avoiding migration of the device of the invention either internally or externally. In some embodiments said patch component further comprises an extension concealing the sclera penetration point, which prevents leak around the tubular component in the early point of the sclera post operation and implantation procedure which in other solutions might lead to severe hypotony immediately following the procedure and during the initial follow up period.

The invention further provides a kit comprising: a tubular component comprising at least one tube; wherein said tubular component has an elongated body having a first end that is positionable at an anterior chamber of the eye and a second end that is positionable at an extraocular space and having an entry point through the Sclera of said eye; wherein said tubular component is configured to provide a fluid path for draining fluid entering the first end of said tubular component from the anterior chamber to the extraocular space via the second end; wherein said tubular component is formed from at least non-porous non-degradable polymer; at least one patch component positionable under the conjunctiva of the eye formed from at least one porous non-degradable polymer; instructions for use of said kit.

The invention further provides a method for controlling the liquid outflow from the anterior chamber of the eye comprising: providing a device of the invention as disclosed herein above and below; and implanting said device into the eye of a subject so that said tubular component is passed through the Sclera of said eye through an entry point and wherein said first end of said tubular component is positioned at an anterior chamber of the eye and said second end of said tubular component is positioned at an extraocular space; and positioning said patch component under the conjunctiva of the eye and above said tubular component and over said Sclera entry point.

The invention further provides a method for the treatment of glaucoma comprising: providing a device of the invention as disclosed herein above and below; and implanting said device into the eye of a subject so that said tubular component is passed through the Sclera of said eye through an entry point and wherein said first end of said tubular component is positioned at an anterior chamber of the eye and said second end of said tubular component is positioned at an extraocular space; and positioning said patch component under the conjunctiva of the eye and above said tubular component and over said Sclera entry point.

### BRIEF DESCRIPTION OF THE DRAWINGS

The subject matter regarded as the invention is particularly pointed out and distinctly claimed in the concluding portion of the specification. The invention, however, both as to organization and method of operation, together with objects, features, and advantages thereof, may best be understood by reference to the following detailed description when read with the accompanying drawings in which:

FIG. 1 shows an embodiment of the device of the invention.

FIG. 2 shows an embodiment of the device of the invention.

It will be appreciated that for simplicity and clarity of illustration, elements shown in the figures have not necessarily been drawn to scale. For example, the dimensions of some of the elements may be exaggerated relative to other elements for clarity. Further, where considered appropriate, reference numerals may be repeated among the figures to indicate corresponding or analogous elements.

### DETAILED DESCRIPTION OF THE PRESENT INVENTION

In the following detailed description, numerous specific details are set forth in order to provide a thorough understanding of the invention. However, it will be understood by those skilled in the art that the present invention may be practiced without these specific details. In other instances, well-known methods, procedures, and components have not been described in detail so as not to obscure the present invention.

The present invention provides an implantable device for controlling liquid outflow from an anterior chamber of an eye and for treating glaucoma, comprising: a tubular component comprising at least one tube; wherein said tubular component has an elongated body having a first end that is positionable at an anterior chamber of the eye and a second end that is positionable at an extraocular space and having an entry point through the sclera of said eye; wherein said tubular component is configured to provide a fluid path for draining fluid entering the first end of said tubular component from the anterior chamber to the extraocular space via the second end; wherein said tubular component is formed from at least non-porous non-degradable polymer; at least one patch component positionable under the conjunctiva of the eye formed from at least one porous non-degradable polymer.

In some embodiments said extraocular space is selected from the retrobulbar space, the retro orbital space and the intraconal space of the eye.

In some embodiments said at least one non-porous non-degradable polymer is selected from polyurethane, polycarbonate, Poly(ethylene-co-vinyl acetate) vinyl acetate, Poly(methyl methacrylate), Poly(propylene carbonate), Poly(vinylidene fluoride), Polyacrylonitrile, Polycarbomethylsilane, Polylactic acid, Polystyrene, Polyvinylpyrrolidone, poly vinyl alcohol (PVA), polyethylene oxide (PEO), polyurethane, polyvinyl chloride (PVC), hyaluronic acid (HA), polyhydroxybuyrate and its copolymers, Nylon 11, Cellulose acetate, poly(3-hydroxybutyric acid-co-3-hydroxyvaleric acid), poly(DL-lactide), polycaprolactone, and poly(L-lactide) or any combination thereof.

In some embodiments said tubular component has a length of at least 10 mm.

In some embodiments said tubular component has a length of between about 10 mm to about 100 mm. In some embodiments said tubular component has a length of about 10, 15, 20, 25, 30, 35, 40, 45, 50, 55, 60, 65, 70, 75, 80, 85, 90, 95, 100 mm.

In some embodiments said tubular component has a lumen diameter of at least 50 micron.

In some embodiments said tubular component has a lumen diameter of between about 50 micron to about 150 micron. In some embodiments said tubular component has a lumen diameter of about 50, 55, 60, 65, 70, 75, 80, 85, 90, 95, 100, 110, 120, 130, 140, 150 micron.

In some embodiments said tubular component has a length and lumen diameter according to the Hagen-Poiseuille Law to provide controlled resistance to liquid outflow from an anterior chamber of an eye to the extraocular space.

In some embodiments said at least one patch component is positioned above said tubular component. In other embodiments said at least one patch component is positioned above and physically attached to said tubular component. In other embodiments said at least one patch component further comprises at least one suturing hole.

In some embodiments said at least one patch component has a thickness of less than 100 microns.

In some embodiments said at least one patch component has a thickness of between about 10 to about 100 microns. In some embodiments said at least one patch component has a thickness of about 10, 15, 20, 25, 30, 35, 40, 45, 50, 55, 60, 65, 70, 75, 80, 85, 90, 95, 100 microns.

In some embodiments said at least one porous non-degradable polymer is selected from polycarbonate, poly-L-lactic acid (PLLA), Poly(DL-lactide-co-caprolactone, Poly(ethylene-co-vinyl acetate) vinyl acetate, Poly(methyl methacrylate), Poly(propylene carbonate), Poly(vinylidene fluoride), Polyacrylonitrile, Polycarbomethylsilane, Polylactic acid, Polystyrene, Polyvinylpyrrolidone, poly vinyl alcohol (PVA), polyethylene oxide (PEO), polyurethane, polyvinyl chloride (PVC), hyaluronic acid (HA), polyhydroxybuyrate and its copolymers, Nylon 11, Cellulose acetate, poly(3-hydroxybutyric acid-co-3-hydroxyvaleric acid), poly(DL-lactide), polycaprolactone, and poly(L-lactide) or any combination thereof.

In some embodiments said porous non-degradable polymer is non-woven.

In some embodiments said porous non-degradable polymer comprises nanofibers.

In some embodiments said porous non-degradable polymer is electrospun.

Electrospun fibers are typically several orders in magnitude smaller than those produced using conventional spinning techniques. By optimizing parameters such as: i) the intrinsic properties of the solution including the polarity and surface tension of the solvent, the molecular weight and conformation of the polymer chain, and the viscosity, elasticity, and electrical conductivity of the solution; and ii) the operational conditions such as the strength of electric field, the distance between spinneret and collector, and the feeding rate of the solution, electrospinning is capable of generating fibers as thin as tens of nanometers in diameter. Additional parameters that affect the properties of electrospun fiber include the molecular weight, molecular-weight distribution and structure (branched, linear etc.) of the polymer, solution properties (viscosity, conductivity and surface tension), electric potential, flow rate and concentration, distance between the capillary and collection screen, ambient parameters (temperature, humidity and air velocity in the chamber), motion of target screen (collector) and so forth. Fabrication of highly porous fibers may be achieved by electrospinning the jet directly into a cryogenic liquid. Well-defined pores developed on the surface of each fiber as a result of temperature-induced phase separation between the polymer and the solvent and the evaporation of solvent under a freeze-drying condition.

Several approaches have been developed to organize electrospun fibers into aligned arrays. For example, electrospun fibers can be aligned into a uniaxial array by replacing the single-piece collector with a pair of conductive substrates separated by a void gap. In this case, the nanofibers tend to be stretched across the gap oriented perpendicular to the edges of the electrodes. It was also shown that the paired electrodes could be patterned on an insulating substrate such as quartz or polystyrene so the uniaxially aligned fibers could be stacked layer-by-layer into a 3D lattice. By controlling the electrode pattern and/or the sequence for applying high voltage, it is also possible to generate more complex architectures consisting of well-aligned nanofibers.

Electrospun nanofibers could also be directly deposited on various objects to obtain nanofiber-based constructs with well-defined and controllable shapes. In addition, one can manually process membranes of aligned or randomly oriented nanofibers into various types of constructs after electrospinning: for example, fabrication of a tube by rolling up a fibrous membrane or the preparation of discs with controllable diameters by punching a fibrous membrane.

The present invention relates to any eletrospinning technique known in the art, which includes Electrospinning, J. Stanger, N. Tucker, and M. Staiger, I-Smithers Rapra publishing (UK), An Introduction to Electrospinning and Nanofibers, S. Ramakrishna, K. Fujihara, W-E Teo, World Scientific Publishing Co. Pte Ltd (Jun 2005), Electrospinning of micro- and nanofibers: fundamentals and applications in separation and filtration processes, Y. Fillatov, A. Budyka, and V. Kirichenko (Trans. D. Letterman), Begell House Inc., New York, USA, 2007, which are all incorporated herein by reference in their entirety.

Suitable electrospinning techniques are disclosed, e.g., in International Patent Application, Publication Nos. WO 2002/049535, WO 2002/049536, WO 2002/049536, WO 2002/049678, WO 2002/074189, WO 2002/074190, WO 2002/074191, WO 2005/032400 and WO 2005/065578, the contents of which are hereby incorporated by reference. It is to be understood that although the according to the presently preferred embodiment of the invention is described with a particular emphasis to the electrospinning technique, it is not intended to limit the scope of the invention to the electrospinning technique. Representative examples of other spinning techniques suitable for the present embodiments include, without limitation, a wet spinning technique, a dry spinning technique, a gel spinning technique, a dispersion spinning technique, a reaction spinning technique or a tack spinning technique. Such and other spinning techniques are known in the art and disclosed, e.g., in U.S. Pat. Nos., 3,737,508, 3,950,478, 3,996,321, 4,189,336, 4,402,900, 4,421,707, 4,431,602, 4,557,732, 4,643,657, 4,804,511, 5,002,474, 5,122,329, 5,387,387, 5,667,743, 6,248,273 and 6,252,031 the contents of which are hereby incorporated by reference.

In some embodiments said porous non-degradable polymer has pores of less than 5 microns.

In some embodiments said porous non-degradable polymer has pores of between about 0.01 microns to about 5 microns. In some embodiments said porous non-degradable polymer has pores of about 0.01, 0.05, 0.1, 0.15, 0.20, 0.25, 0.30, 0.35, 0.40, 0.45, 0.50, 0.55, 0.60, 0.65, 0.70, 0.75, 0.80, 0.85, 0.90, 0.95, 1.0, 1.5, 2.0, 2.5, 3.0, 3.5, 4.0, 4.5, 5 micron.

In some embodiments said at least one patch component further comprises at least one pharmaceutical active agent.

In some embodiments said at least one patch component further comprises at least one pharmaceutical active agent selected from: an antifibrotic agent, an anticoagulant, an antithrombotic agent, an antibiotic agent, an anti-inflammatory agent, a biodegradable material, a pressure lowering agent, an antiangiogenic agent or any combination thereof.

The invention further provides a kit comprising: a tubular component comprising at least one tube; wherein said tubular component has an elongated body having a first end that is positionable at an anterior chamber of the eye and a second end that is positionable at an extraocular space and having an entry point through the Sclera of said eye; wherein said tubular component is configured to provide a fluid path for draining fluid entering the first end of said tubular component from the anterior chamber to the extraocular space via the second end; wherein said tubular component is formed from at least non-porous non-degradable polymer; at least one patch component positionable under the conjunctiva of the eye formed from at least one porous non-degradable polymer; instructions for use of said kit.

The invention further provides a method for controlling the liquid outflow from the anterior chamber of the eye and for treating glaucoma comprising: providing a device of the invention as disclosed herein above and below; and implanting said device into the eye of a subject so that said tubular component is passed through the Sclera of said eye through an entry point and wherein said first end of said tubular component is positioned at an anterior chamber of the eye and said second end of said tubular component is positioned at an extraocular space; and positioning said patch component under the conjunctiva of the eye and above said tubular component and over said Sclera entry point.

In another one of its aspects the preset invention provides a surgical method for implanting a device of the invention into the eye of a subject, said method comprising: performing a localized peritomy comprising removing conjunctiva and tenon capsule; creating a trans-scleral tubular channel; in some embodiments said tubular channel is created by means of a sharp cylindrical knife; locating said device into said tubular channel wherein at least one end of said device is directed towards (or located at) the anterior chamber and its opposite end is directed towards (or located at) the extra orbital space; covering the entry point of the sclera of the eye with said patch component and in some embodiments suturing said patch.

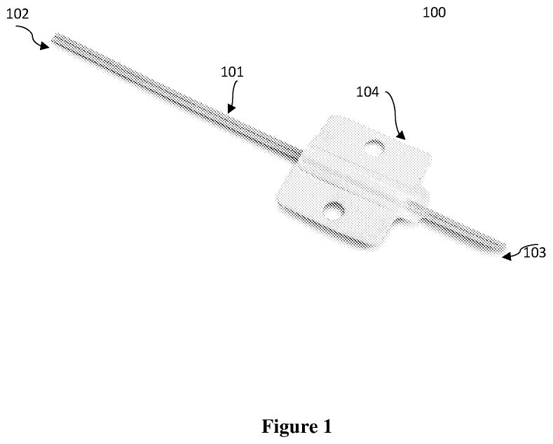

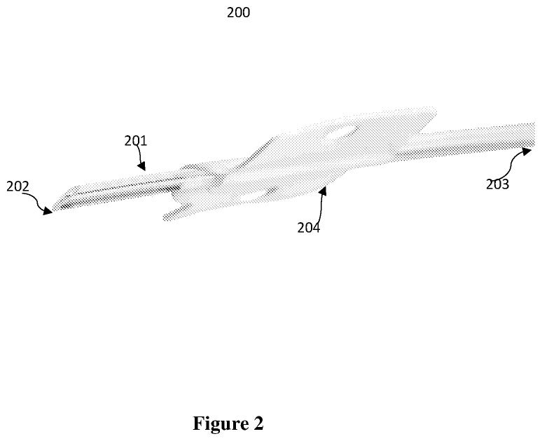

FIGS. 1 and 2 show embodiments of an implantable device for controlling liquid outflow from an anterior chamber of an eye of the invention.

FIG. 1 shows the device (100) comprising a tubular component comprising at least one tube (101) wherein said tubular component has an elongated body having a first end (102) that is positionable at an anterior chamber of the eye and a second end (103) that is positionable at an extraocular space and having an entry point through the sclera of said eye; wherein said tubular component is configured to provide a fluid path for draining fluid entering the first end of said tubular component from the anterior chamber to the extraocular space via the second end; wherein said tubular component is formed from at least non-porous non-degradable polymer; at least one patch component (104) positionable under the conjunctiva of the eye formed from at least one porous non-degradable polymer.

FIG. 2 shows another view of a device of the invention (200) comprising a tubular component comprising at least one tube (201) wherein said tubular component has an elongated body having a first end (202) that is positionable at an anterior chamber of the eye and a second end (203) that is positionable at an extraocular space and having an entry point through the sclera of said eye; wherein said tubular component is configured to provide a fluid path for draining fluid entering the first end of said tubular component from the anterior chamber to the extraocular space via the second end; wherein said tubular component is formed from at least non-porous non-degradable polymer; at least one patch component (204) positionable under the conjunctiva of the eye formed from at least one porous non-degradable polymer.

While certain features of the invention have been illustrated and described herein, many modifications, substitutions, changes, and equivalents will now occur to those of ordinary skill in the art. It is, therefore, to be understood that the appended claims are intended to cover all such modifications and changes as fall within the true spirit of the invention.

1. An implantable device for controlling liquid outflow from an anterior chamber of an eye, comprising:
a tubular component comprising at least one tube; wherein said tubular component has an elongated body having a first end that is positionable at an anterior chamber of the eye and a second end that is positionable at an extraocular space and having an entry point through the sclera of said eye;
wherein said tubular component is configured to provide a fluid path for draining fluid entering the first end of said tubular component from the anterior chamber to the extraocular space via the second end; wherein said tubular component is formed from at least non-porous non-degradable polymer;
at least one patch component positionable under the conjunctiva of the eye formed from at least one porous non-degradable polymer.

2. An implantable device according to claim 1, wherein said extraocular space is selected from the retrobulbar space, the retro orbital space and the intraconal space of the eye.

3. An implantable device according to claim 1, wherein said at least one non-porous non-degradable polymer is selected from polyurethane, polycarbonate, Poly(ethylene-co-vinyl acetate) vinyl acetate, Poly(methyl methacrylate), Poly(propylene carbonate), Poly(vinylidene fluoride), Polyacrylonitrile, Polycarbomethylsilane, Polylactic acid, Polystyrene, Polyvinylpyrrolidone, poly vinyl alcohol (PVA), polyethylene oxide (PEO), polyurethane, polyvinyl chloride (PVC), hyaluronic acid (HA), polyhydroxybuyrate and its copolymers, Nylon 11, Cellulose acetate, poly(3- hydroxybutyric acid-co-3-hydroxyvaleric acid), poly(DL-lactide), polycaprolactone, and poly(L-lactide) or any combination thereof.

4. An implantable device according to claim 1, wherein said tubular component has a length of at least 10 mm.

5. An implantable device according to claim 1, wherein said tubular component has a length of between about10 mm to about 100 mm.

6. An implantable device according to claim 1, wherein said tubular component has a lumen diameter of at least 50 micron.

7. An implantable device according to claim 1, wherein said tubular component has a lumen diameter of between about 50 micron to about 150 micron.

8. An implantable device according to claim 1, wherein said tubular component has a length and lumen diameter according to the Hagen-Poiseuille Law to provide controlled resistance to liquid outflow from an anterior chamber of an eye to the extraocular space.

9. An implantable device according to claim 1, wherein said at least one patch component is positioned above said tubular component.

10. An implantable device according to claim 1, wherein said at least one patch component has a thickness of less than 100 microns.

11. An implantable device according to claim 1, wherein said at least one patch component has a thickness of between about 10 to about 100 microns.

12. (canceled)

13. An implantable device according to claim 1, wherein said porous non-degradable polymer is non-woven.

14. An implantable device according to claim 1, wherein said porous non-degradable polymer comprises nanofibers.

15. An implantable device according to claim 1, wherein said porous non-degradable polymer is electrospun.

16. An implantable device according to claim 1, wherein said porous non-degradable polymer has pores of less than 5 microns.

17. An implantable device according to claim 1, wherein said porous non-degradable polymer has pores of between about 0.01 microns to about 5 microns.

18. An implantable device according to claim 1, wherein said at least one patch component further comprises at least one pharmaceutical active agent.

19. An implantable device according to claim 1, wherein said at least one patch component further comprises at least one pharmaceutical active agent selected from: an antifibrotic agent, an anticoagulant, an antithrombotic agent, an antibiotic agent, an anti-inflammatory agent, a biodegradable material, a pressure lowering agent, an antiangiogenic agent or any combination thereof.

20. A kit comprising an implantable device according to claim 1 and instructions for use of said kit.

21. A method for controlling the liquid outflow from the anterior chamber of the eye comprising:
providing an implantable device according to claim 1, and
implanting said device into the eye of a subject so that said tubular component is passed through the Sclera of said eye through an entry point and wherein said first end of said tubular component is positioned at an anterior chamber of the eye and said second end of said tubular component is positioned at an extraocular space; and
positioning said patch component under the conjunctiva of the eye and above said tubular component and over said Sclera entry point.
